(19) United States
(12) Patent Application Publication
(10) Pub. No.: US 2010/0217168 A1
(43) Pub. Date: Aug. 26, 2010

(54) Orthosis

(76) Inventors: Marcus James King, Christchurch (NZ); Julian Verkaalk, Christchurch (NZ); Lan Le-Ngoc, Christchurch (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/089,227

(22) PCT Filed: Jun. 23, 2006
(86) PCT No.: PCT/NZ06/00160

§ 371 Date: Apr. 4, 2008

(30) Foreign Application Priority Data
Jun. 23, 2005 (NZ) 540912

(51) Int. Cl. A61F 5/00

(52) U.S. Cl. 602/21

(57) ABSTRACT

A hand orthosis for assisting gripping by persons with a disability affecting hand function, comprises a thumb part, a hand part which fits around a user's hand between the thumb and knuckles, a forearm part which fits to a user's forearm above the hand. The orthosis includes a flexible connecting element connected at one end to the thumb part and which is movably coupled to the hand part and connected at another end to the forearm part such that when the orthosis is fitted to a user, extension of the user's wrist draws the user's thumb towards the user's index finger to form a grip. A powered embodiment of the orthosis is also disclosed.

### FIELD OF THE INVENTION

The invention relates to orthoses, which are devices that are attached to the outside of a limb to provide support to or assist the motion of the limb, and in particular to hand orthoses to assist gripping by persons with a disability affecting hand function.

### BACKGROUND TO THE INVENTION

It is desirable to provide some function of the hand to those suffering disabilities that affect hand function. Many persons with tetraplegia would rather regain hand function than many other bodily functions including the ability to walk.

One example class of persons suffering disabilities that affect hand function are those with C5 tetraplegia. Persons in this class may have some arm function, such as in the bicep or lower arm, but may have lost the function of their wrists or fingers. Such persons may have little or no effective grip.

A known surgical method to address the above problem involves a procedure of tendon transfer. This procedure involves removal of the tendon from disabled muscles and attachment of the removed tendon to a functional muscle such as the brachioradialis. This enables the subject to then perform wrist extensions. Another procedure involves the removal of the tendon of the thumb and anchoring onto the radialis bone of the lower arm. After this procedure, when a person with C5 tetraplegia extends their wrist, the anchored or fixated thumb adducts onto the opposing index finger and forms a tendonesis grip.

U.S. Pat. No. 4,258,441 discloses a hand and lower arm prosthesis for amputees who have lost a hand and lower arm, in which shoulder movement is transferred through a cable to enable a grip to be formed between the thumb and fingers of the hand prosthesis.

U.S. Pat. No. 5,222,986 discloses a prosthesis which replaces lost fingers and which enables a mechanical grip to be formed to grasp objects, via wrist movement.

U.S. Pat. No. 5,409,447 discloses a glove with external cords for operating the fingers and thumb of a disabled hand. The user's fingers must be inserted into the glove and the glove fits over all of the fingers of the disabled hand, and cords are pulled to close the fingers.

U.S. Pat. No. 4,167,044 discloses a means for actuating a disabled hand including a number of ring-like members through which disabled fingers are inserted. Cables are connected to the ring-like members which when pulled move the disabled fingers.

It is an object of the invention to provide an improved orthosis device or at least to provide disabled persons and their physicians or therapists with a useful choice.

### SUMMARY OF THE INVENTION

As used herein, the term ‘key pinch’ in relation to grip refers to a grip formed by drawing the thumb towards the index finger and in particular the side of the index finger, to either close the thumb pad against the side of the index finger, or move the thumb sufficiently close to the index finger to enable an object to be gripped between the thumb pad and the side of the index finger.

In broad terms an aspect of the invention comprises a hand orthosis comprising;
a thumb part attachable to a user's thumb
a hand part fittable around a user's hand between the thumb and knuckles,
a forearm part fittable to a user's forearm above the hand, and
a flexible connecting element connected at one end to the thumb part and which is moveably coupled the hand part and connected at another end to the forearm part such that when the orthosis is fitted to a user, extension of the user's wrist draws the user's thumb towards the user's index finger to form a key grip.

In broad terms in another aspect the invention comprises a hand orthosis comprising;
a thumb part attachable to a user's thumb
a hand part fittable around a user's hand without insertion of the user's fingers into and covering of the user's fingers by the orthosis,
a forearm part fittable to a user's forearm above the hand, and
a flexible connecting element connected at one end to the thumb part and which is movably coupled to the hand part and connected at another end to the forearm part such that when the orthosis is fitted to a user, extension of the user's wrist can draw the user's thumb against the user's index finger to form a grip.

In broad terms in a further aspect the invention comprises a hand orthosis comprising;
a thumb part attachable to a user's thumb
a hand part for fittable around a user's hand between the thumb and knuckles,
a forearm part for fittable to a user's forearm above the hand, and
a connecting element connected to the thumb part and to another part of the orthosis,
a power unit to which the connecting element is also connected, and
a control device operable by the user to cause the power unit to via the connecting element draw the user's thumb towards the user's index finger to form a grip

Preferably the orthosis comprises one or more fasteners for releasably securing the forearm part around a user's forearm, which preferably enable the forearm part to be opened so that the forearm part can be fitted to or removed from a user's forearm without requiring sliding of the user's forearm part through the forearm part. Preferably the one or more fastener(s) of the forearm part is/are on the outside of the user's forearm when the orthosis is worn.

In one embodiment the thumb part is in the form of C-shaped clip formed of a resiliently flexible material.

The term ‘comprising’ as used in this specification means ‘consisting at least in part of’, that is to say when interpreting statements in this specification which include that term, the features, prefaced by that term in each statement, all need to be present but other features can also be present.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

This invention may also be said broadly to consist in the parts, elements and features referred to in this specification, individually or collectively, and any combinations of any two or more said parts, elements or features. Where specific integers are mentioned herein which have known equivalents in the art to which this invention relates, such known equivalents are deemed to be incorporated herein as if individually set forth.

### BRIEF DESCRIPTION OF THE FIGURES

Preferred forms of orthosis device of the invention are described by way of example with reference to the accompanying figures in which:

FIG. 1 is a view of a preferred form hand orthosis with securing straps thereof fully undone and with the orthosis laid out flat, from one side,

FIG. 2 is a view of the preferred form hand orthosis laid out flat and similar to FIG. 1, but from the other side of the orthosis,

FIG. 3 is view of the orthosis worn by a user, from the inside arm side (and in which the user has not extended his or her wrist to form a grip),

FIG. 4 is a view of the orthosis worn by a user, from the top side of the user's forearm,

FIG. 5 is a view of the orthosis worn by a user, from the underside of the user's forearm,

FIG. 6 is a view of the orthosis worn by a user, from the back side of the user's forearm,

FIG. 7 is a view of the orthosis worn by a user with the user's wrist in a relaxed position so that the user's thumb is not closed against the user's fingers to form a grip,

FIG. 8 is a view similar to FIG. 7 but showing the user's wrist extended to close the user's thumb against the user's fingers and form a grip, and showing the user gripping a pen,

FIG. 9 schematically shows a preferred form of a powered orthosis of the invention,

FIG. 10 shows the preferred form powered orthosis in use, and

FIG. 11 shows an orthosis having an additional strap for assisting maintaining a user's fingers closed when forming a grip.

### DETAILED DESCRIPTION OF PREFERRED FORMS

The orthosis of the invention comprises a thumb part 1, hand part 2, and forearm part 3.

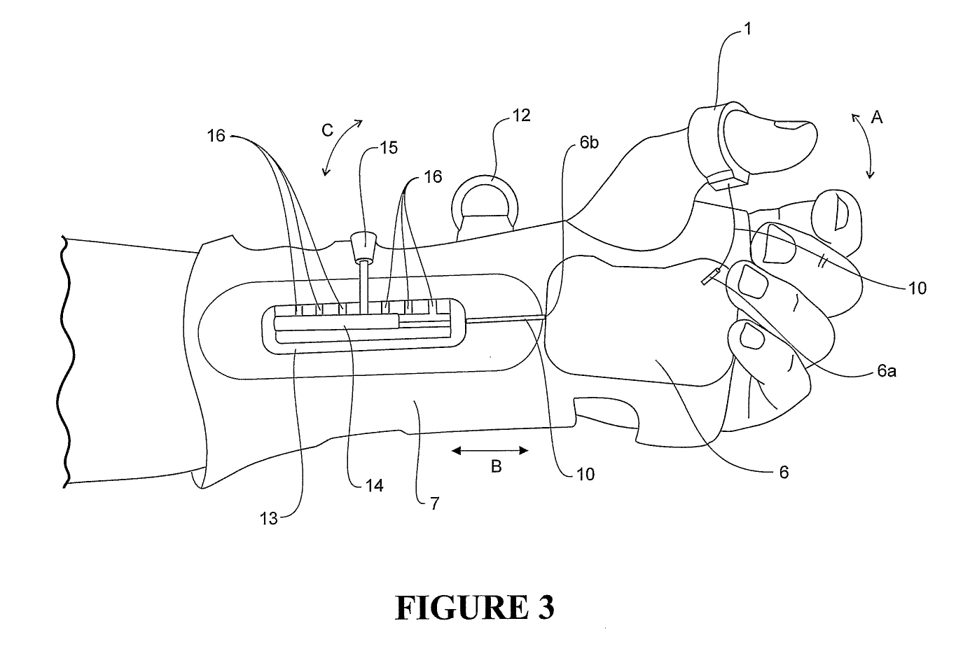

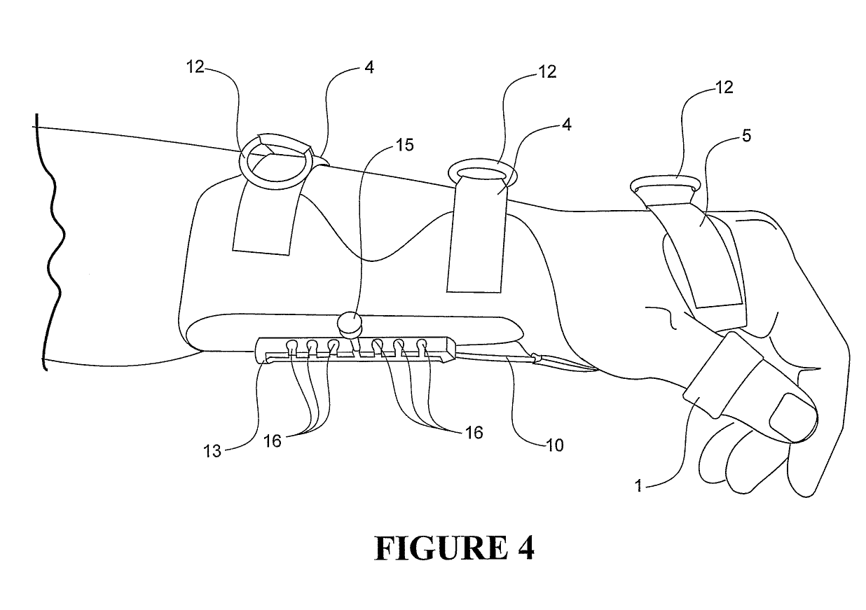

The thumb part 1 in the preferred form comprises a C-shaped clip formed of a resiliently flexible material such as a resilient plastics material for example. When the orthosis is worn the thumb part is attached to the user's thumb. In the preferred form the thumb part 1 is designed to fit over and onto the middle phalange of the user's thumb as shown in for example FIGS. 3 and 4. The thumb part 1 may be made of a thermoformable plastics material so that it can be shaped to fit a particular user after heating with hot water for example (and retain its modified shape on cooling) or it may be made from a metal such as copper that can be bent to fit the user's thumb and thereafter retains its shape. In an alternative form the thumb part may comprise a strip of flexible material such as a soft webbing material for example, with sections of hook and loop fastening material attached, which may be wrapped and fastened around the user's thumb.

The forearm part 3 includes two adjustable straps 4 for securing the orthosis to a user's forearm above the hand, and the hand part 2 includes strap 5 for fitting around a user's hand, as will be further described.

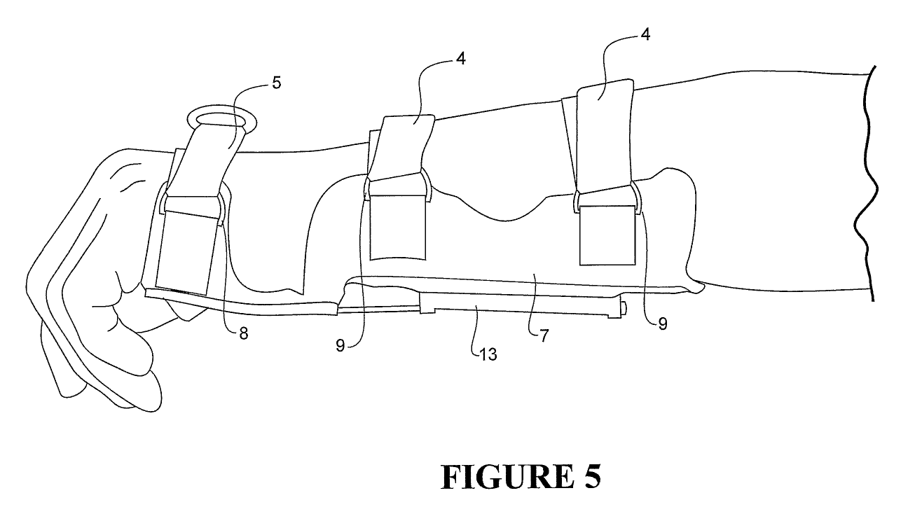

FIGS. 3 to 6 show the preferred form orthosis fitted to or worn by a user. The hand part 2 fits around the user's hand. Palm section 6 fits over the palm of the hand between the thumb and knuckles. Strap 5 extends from one side of the palm section 6 and around the back of the hand to the other side of the palm section 6, so that the hand part 2 including the strap 5 fits around or encircles the user's hand as shown. In the preferred form shown the palm section 6 substantially covers the palm of the user's hand but in an alternative form the palm section 6 or equivalent of the hand part 2 may be of reduced size. The hand part of the orthosis is not formed as a glove requiring that the user insert his or her fingers into a glove-like orthosis, in order to fit the orthosis to the user's hand.

The forearm part 3 of the orthosis comprises a major section 7 which when the orthosis is worn extends longitudinally of the user's inside forearm as shown particularly in FIG. 3. The forearm part 3 also includes the straps 4, which when the orthosis is worn extend from one side of the forearm section 7 to the opposite side around the back of the user's forearm, as shown in FIGS. 3 to 6.

Thus when the preferred form orthosis is worn as shown, the orthosis comprises palm and inside forearm sections 6 and 7 with straps 4 and 5 which encircle the lower forearm and the hand. The preferred embodiment orthosis is simply strapped on as is further described, and does not comprise a full or part glove with finger parts into which a user's fingers must be inserted to fit the orthosis.

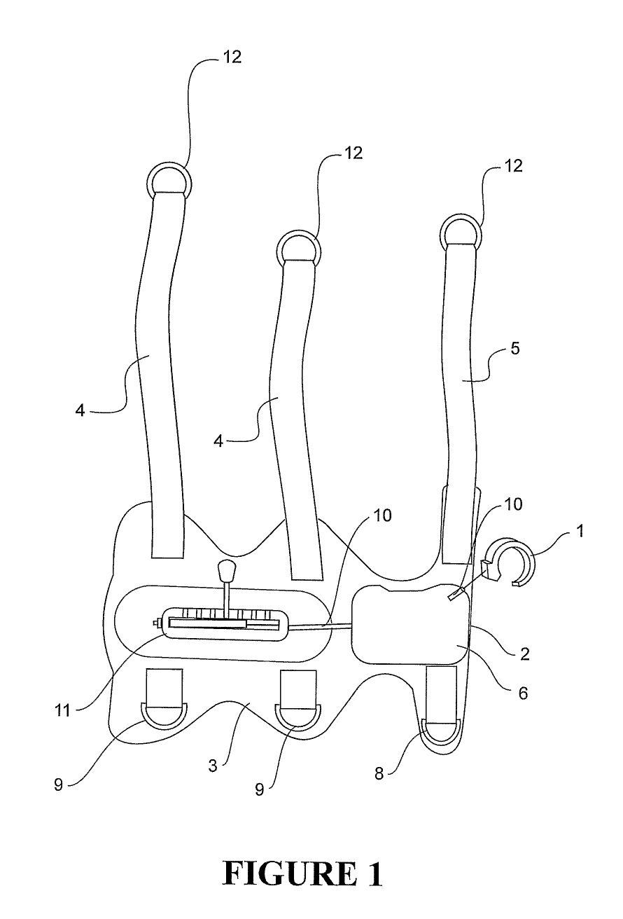

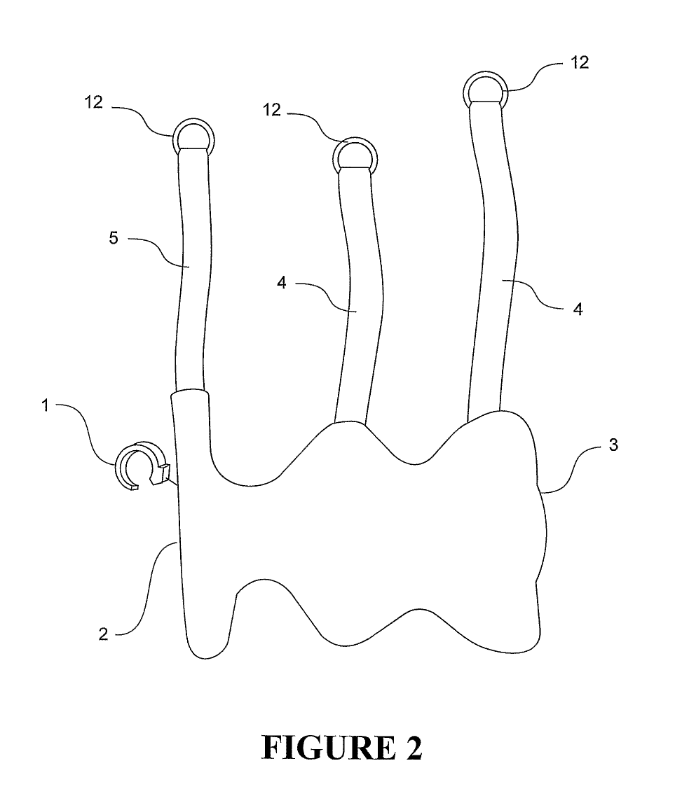

In the preferred form, typically the palm and forearm section 6 and 7 are formed of a flexible material such as a heavyweight flexible fabric material or alternatively a flexible planar foam material such as Neoprene for example. The straps 4 and 5 may be formed from a webbing material for example. FIG. 1 shows the outside of the orthosis (outside when the orthosis is worn) and FIG. 2 the inside of the orthosis, laid out flat with the straps fully undone. The inside of the palm and forearm sections 6 and 7 may be lined with for example a softer fabric material or a cloth backed light foam material if desired, so that the orthosis is as comfortable as possible to the wearer.

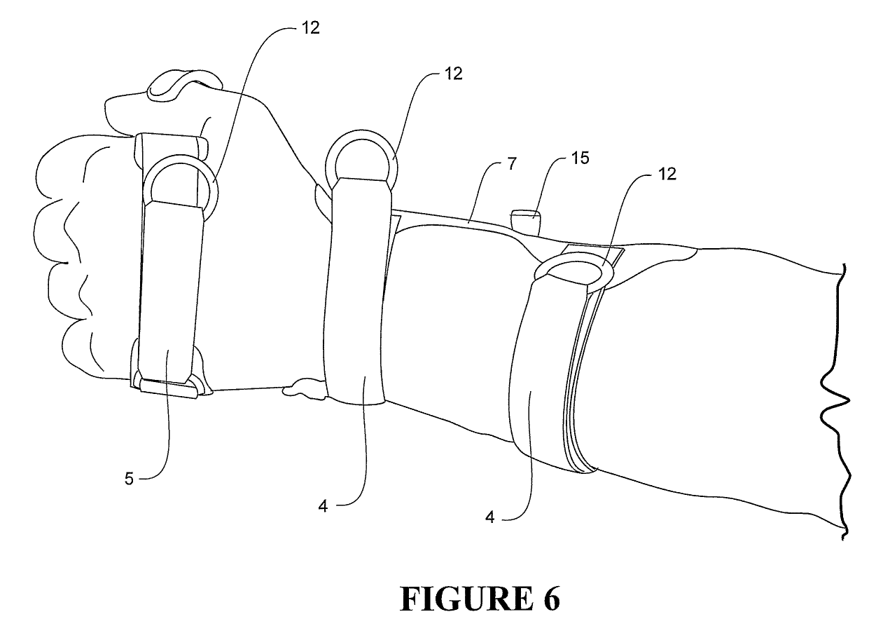

In the preferred form orthosis the straps 4 and 5 pass through D-rings on the opposite side of the orthosis, and is then doubled back on themselves when the orthosis is worn, and sections of a hook and loop fastening material are attached to the straps 4 and 5, so that the far ends of the straps may be attached back on themselves to secure the straps. In particular strap 5 passes around the hand of the user and through the D-ring 8, and then the far end of the strap 5 is attached back on itself to secure the hand part of the orthosis, as shown particularly in FIG. 6. Straps 4 pass through respective D-rings 9 and double back and are attached back to themselves, again as in particular shown in FIG. 6. This arrangement has been found the easiest for disabled persons to use, but alternatively the straps 4 and 5 could be fastened by other clip or buclde arrangements such as plastic plug-in clips or similar, preferably also allowing for adjustment in length to fit the orthosis correctly. Alternatively again straps 4 securing the orthosis in place may extend at an angle (or diagonally) around the forearm from one side of the forearm part 7 of the orthosis to the other, in a lacing arrangement. For example a single strap may crisscross back and forth around the user's forearm at a number of points, or two straps may crisscross each other and between opposite sides of the forearm part 7 or equivalent. In a further alternative arrangement the forearm part may wrap completely around the user's forearm or in particular lower part thereof, and be closed by a zipper which extends longitudinally of the orthosis and forearm. The orthosis may be fitted by placing the open orthosis on the user's forearm and closing the zip, and vice versa to remove the othosis.

Cable 10 is connected at one end to the thumb part 1 and passes through the hand part 2 and is connected to the forearm part 3. The cable 10 is moveably connected to the hand part 2, and then connects to the forearm part 3. In the preferred form the cable 10 passes beneath the material of the palm section 6 but could alternatively pass through a small plastic ring or fabric loop attached to the hand part 2. The cable enters the palm section at 6a, and exits the section of the palm section 6b. In the preferred form the other end of the cable 10 is connected to to a cable adjustment device generally indicated at 11.

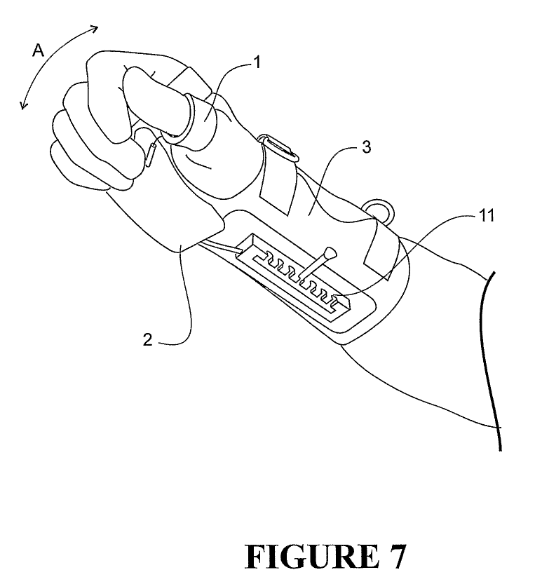

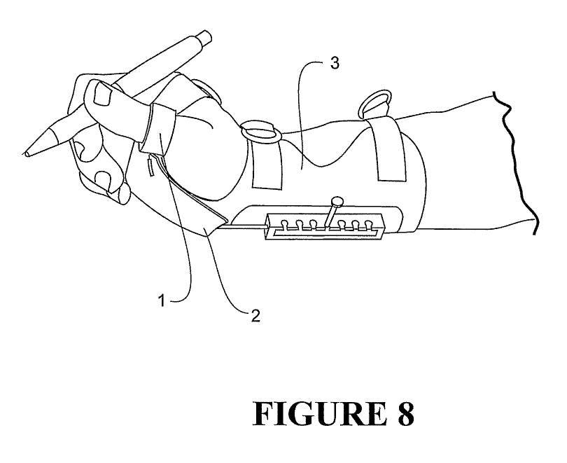

FIG. 7 shows the orthosis worn by a user, with the user's wrist at rest, that is, not in extension. In the position shown, the user's fingers and thumb cannot form an effective grip because the user is affected by a disability preventing the forming of a grip. By contrast in FIG. 8, using the orthosis the user has formed a grip and in particular a key pinch grip. To form a grip, the user extends or cocks back his or her wrist in the direction of arrow A in FIG. 7 to the position shown in FIG. 8. This extension of the user's wrist increases the distance between the user's finger(s)/thumb and the user's forearm when measured along the path of the cable 10. Due to the fixed length of the cable and its attachment between the thumb part 1 and forearm part 3 of the orthosis through the palm section 6, extension of the user's wrist draws the thumb toward the side of the user's index finger to form a key pinch grip. FIG. 8 shows a wearer holding a pen via the resulting key pinch grip. In use, a person who is normally unable to form an effective grip due to disability, may use the orthosis device to grip items, such as a cup or a pen, by arranging the item between the user's thumb and finger(s), and extending his or her wrist. To release the grip, the user relaxes the wrist extension, which removes the pulling force of the cable on the user's finger(s)/thumb and consequently removes the grip.

In the description thus far the connection between the thumb part 1 and forearm part 7 through the palm section 6 is referred to as a cable but this term is used in a general sense and any suitable flexible connecting element may be employed such as a single strand or monofilament plastics element, a single or multi-stranded wire, or similar. The term “cable” is to be understood accordingly.

A particular advantage of the orthosis of the invention is that the hand part of the orthosis fits around the user's hand only between the thumb and knuckles. In the preferred form shown, this is by strap 5 extending around the back of the hand from one side of the palm section 6 to the other. The hook and loop fastening system (or equivalent) of the strap 5 enables adjustment of the size of the internal diameter of the hand part when closed, so that the orthosis may be used with people with different sizes of hand but alternatively the orthosis may come in a range of sizes so that adjustment of the size of the hand part via the adjustment of the strap 5 is not necessary. The orthosis does not take the form of a glove, or comprise glove fingers into which a user's fingers must be inserted in order to fit the orthosis. It is very difficult for a person with a disabled hand with minimal or no finger movement, to fit his or her fingers into a glove-like orthosis. A major benefit of the preferred form orthosis of the invention is that some disabled persons may be able to fit the orthosis without requiring assistance from a third party. To do so the user may place the orthosis on a flat surface with the inside of the orthosis up, so that the orthosis is laid out as shown in FIG. 2. The user then simply lies his or her disabled hand and forearm on the orthosis, and then with his or her other arm passes each of the straps over the disabled hand and arm and through the D-rings 8 and 9 to fix the orthosis in place. The user also fits the thumb clip 1 to the user's disabled thumb. Alternatively it has been found that after the orthosis has been worn once or a few times by a user to whom the orthosis has been fitted with the assistance of another person, that even where the user has two disabled hands/arms in some cases the user may undo the straps 4 and 5 with his or her mouth when removing the orthosis, and after inserting his or her arm into the loosened orthosis may also retighten the straps 4 and 5 by mouth. That is, the user may grasp the soft plastic rings 12 provided on the ends of the straps 4 and 5 in his or her teeth (see FIG. 6) and peel back the ends of the straps, to loosen the orthosis so that the user may then remove his or her hand and arm from the orthosis. If the user then leaves the orthosis straps loosened but without pulling the straps back through the D-rings 8 and 9, then when the user goes to use the orthosis again another time, some users may be able to insert his or hand and arm through the orthosis with the straps loosened, and when the orthosis is roughly in place then again grip the free ends of the straps 4 and 5 in their teeth, tension them, and stick them down back on themselves (by a hook and loop fastening system) to secure the orthosis in place. For those users who can use the orthosis in this way, being able to do so is an advantage over prior art orthoses which typically require fitting and removal of the orthosis with the assistance of a third party. To assist this, the ends of the straps 4 and 5 may be enlarged, and in the preferred form are formed as flexible rubber or plastic rings 12. These are larger than the D-rings 8 and 9 through which the ends of the straps must be passed, but are compressible or foldable to facilitate this.

The preferred form orthosis includes a cable adjustment device shown particularly in FIG. 3. The adjustment device comprises a plastic barrel 13 fixed to the inside arm of the orthosis and particularly to the forearm section 7. The end of the cable 10 is a fixed to a bolt 14 which is slidably moveable in the barrel 13 in the direction of arrow B in FIG. 3. A laterally extending lever 15 fixed to the bolt 14 intermediate of its length can be moved in the direction of arrow C in FIG. 3 to release the lever from one of a series of notches 16, to enable the bolt 14 to then be moved in the barrel 13 to loosen or tighten the cable 10. This enables adjustment of the degree of closure between the pad of the user's thumb and the side of the index finger when the wrist is extended to form a key pinch grip formed via the orthosis. When the bolt 14 is moved rearwardly to its maximum extent (towards the elbow), a relatively tight grip will be formed between the thumb pad and index finger directly. When the bolt is moved forwardly a looser grip is formed, enabling larger objects to be picked up. This bolt mechanism enables the user to adjust his or her grip for broadening the range of items that the user may be able to grip or pick up, from a key to a pen to a book for example, with no assistance from an able bodied helper. The bolt allows the user to adjust the cable length without assistance even though they may have little or no function in either hand. Being able to adjust the orthosis independently in this way is an advantage over prior art orthoses which typically require a third party to adjust them.

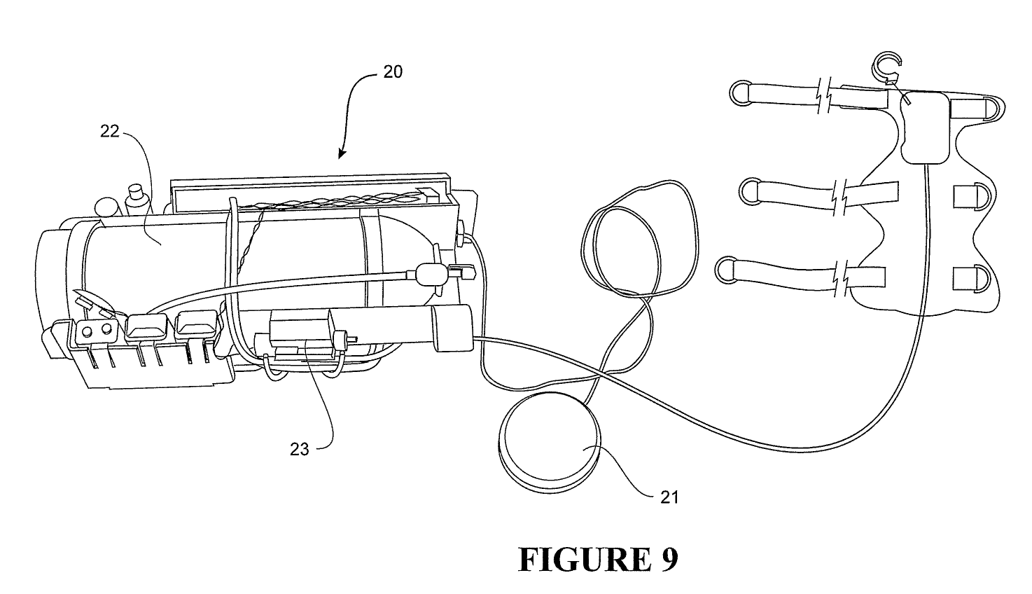

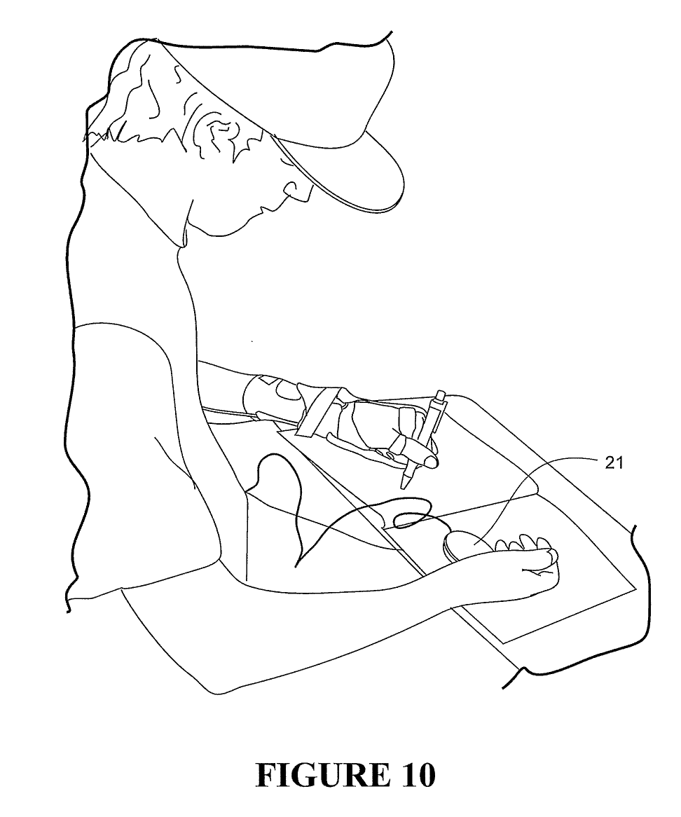

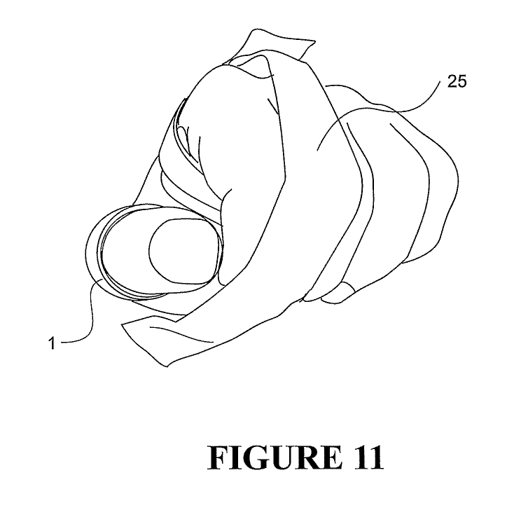

The orthosis of FIGS. 1 to 8 and described thus far is a manual orthosis. That is, wrist extension action by a wearer of the orthosis causes the wearer's thumb to move towards or close against the index finger to form a key grip. FIG. 9 schematically shows a preferred form of a powered orthosis and FIG. 10 shows a powered version of the orthosis in use. The orthosis as worn by the user is the same as the manual orthosis described previously and as shown in FIGS. 1 to 6, with the difference that the cable instead of connecting to the forearm part 3 of the orthosis and in particular the cable adjustment mechanism 11 of the preferred form manual orthosis, connects to a power unit generally indicated at 20 in FIG. 9. Also in this embodiment the cable 10 is a Bowden-type cable. The near end of the inner cable connects at one end to the thumb part 1 as before and passes through the palm section 6 as described previously. The far end of the inner cable and outer sheath connect to an actuator of the power unit. The near end of the outer sheath of the Bowden cable extends from the actuator to the palm section of the orthosis and is fixed to the orthosis. When the power unit is actuated the Bowden cable moves the thumb clip 1 to close and open a key grip. The user also has a switch 21 for actuating the power unit. The switch may be positioned so that it can be actuated by any mobile region of the user's body, such as a shoulder or head or the other arm. Sequential operation of the switch causes the actuator to open or close the key grip in a cycle. The user positions their thumb and forefinger around the object that is desired to be manipulated and then operates the switch 21 to form the key-pinch grip. When it is desired to release the object the switch 21 is again operated. The powered orthosis enables a disabled person to form a grip for an extended period, enabling the person to hold a pen while writing over a number of minutes as shown in FIG. 11 for example. The forearm and palm sections of the orthosis may incorporate a wrist splint that stiffens the hand and forearm to ensure that the wrist does not bend when a force is applied to an object that is manipulated. This stiffener could be made from theromoformable plastic that may be a standard shape or may be custom shaped to fit the user.

The power unit may be any form of actuator that tensions a Bowden cable on demand. Such power units include pneumatic or hydraulic cylinders or electric motors (rotary or linear) or shape changing alloys or polymers that change shape when an electric voltage is applied or some other form of actuator not described here. In the preferred form the power unit 20 is mobile and includes an air accumulator and air cylinder 22, which provides air pressure to actuate a pneumatic ram 23 to which the Bowden cable is connected and which operates the Bowden cable when the switch 21 is actuated by the user. Air pressure to the pneumatic ram 23 is controlled by an electric solenoid operated air valve controlled by switch 21. The powered orthosis also comprises a small battery powered compressor (not shown) that can be connected to the accumulator and cylinder 22 to provide air pressure in the cylinder.

Some persons with minimal or no movement remaining in their hand may find it difficult to close their index and other fingers sufficiently to enable the thumb when drawn towards the index finger to form an effective key grip against the side of the index finger. Referring to FIG. 11 an additional strap 25 of for example webbing material may extend from the hand part 2 of the orthosis. This additional strap 25 may be used, when the orthosis is otherwise in place on the user's hand and forearm, by drawing it around the index and next finger of the user's hand as shown and attaching the far end of the strap 25 back to the orthosis on the palm section 6 or inside arm part 7 of the orthosis, to hold the index finger and next finger closed. The far end of the strap 25 may also be attachable to the inside arm side of the orthosis by a hook and loop fastening system for example.

The foregoing describes the invention including preferred forms thereof. Alterations and modifications as will be obvious to those skilled in the art are intended to be incorporated within the scope hereof as defined in the accompanying drawings.

1. A hand orthosis comprising;
a thumb part attachable to a user's thumb
a hand part fittable around a user's hand between the thumb and knuckles,
a forearm part fittable to a user's forearm above the hand, and
a flexible connecting element connected at one end to the thumb part and which is movably coupled to the hand part and connected at another end to the forearm part such that when the orthosis is fitted to a user, extension of the user's wrist draws the user's thumb towards the user's index finger to form a grip.

2. A hand orthosis comprising;
a thumb part attachable to a user's thumb
a hand part fittable around a user's hand without insertion of the user's fingers into and covering of the user's fingers by the orthosis,
a forearm part fittable to a user's forearm above the hand, and
a flexible connecting element connected at one end to the thumb part, and which is movably coupled to the hand part and connected at another end to the forearm part such that when the orthosis is fitted to a user, extension of the user's wrist can draw the user's thumb against the user's index finger to form a grip.

3. A hand orthosis according to claim 1 wherein the forearm part comprises one or more fasteners for releasably securing the forearm part around a user's forearm.

4. A hand orthosis according to claim 3 wherein the one or more fastener(s) enable the forearm part to be opened so that the forearm part can be fitted to or removed from a user's forearm without requiring sliding of the user's forearm part through the forearm part.

5. A hand orthosis according to claim 4 wherein the one or more fastener(s) of the forearm part is/are on the outside of the user's forearm when the orthosis is worn.

6. A hand orthosis according to claim 5 wherein the forearm part comprises a major section which extends longitudinally of the user's inside forearm when the orthosis is worn, and at least one strap attached thereto which extends from one side to an opposite side of said major section around the back of the user's forearm.

7. A hand orthosis according to claim 5 wherein the forearm part comprises a major section which extends longitudinally of the user's inside forearm part when the orthosis is worn, and two or more straps attached thereto which when the orthosis is worn can extend from one side to another of said major section around the back of the user's forearm.

8. A hand orthosis according to claim 6 wherein when the orthosis is worn said strap(s) can pass from one side of said major section and around the back of the user's forearm to said opposite side of the major section of the forearm part, and then double back on itself/themselves.

9. A hand orthosis according to claim 8 comprising one or more rings or loops attached to said opposite side so that before doubling back the strap(s) may pass through a ring or loop at said opposite side of the major section of the forearm part.

10. A hand orthosis according to claim 9 wherein each strap comprises sections of a hook and loop fastening material by which a far end of the strap may be attached back to itself.

11. A hand orthosis according to claim 8 wherein each strap has an enlarged far end.

12. A hand orthosis according to claim 10 wherein each strap has an enlarged far end which is larger than said ring or loop and is compressible or foldable for passing said enlarged end through said ring or loop.

13. A hand orthosis according to claim 1 wherein said thumb part is in the form of C-shaped clip formed of a resiliently flexible material.

14. A hand orthosis according to claim 1 wherein the hand part comprises a palm section which when the orthosis is worn extends at least partly over the palm of the user's hand, and a strap which can extend from one side of said palm section around the back of the hand to another side of the palm section to encircle the hand.

15. A hand orthosis according to claim 14 wherein said strap of the hand part may pass from one side of said palm section, around the back of the user's hand, and to said another side of said palm section and then double back on itself.

16. A hand orthosis according to claim 15 comprising a ring or loop attached to said another side of said palm section so that before doubling back on itself the strap may pass through said ring or loop.

17. A hand orthosis according to claim 16 wherein said strap of the hand part comprises sections of a hook and loop fastening material by which a far end of the strap may be attached back to itself.

18. A hand orthosis according to claim 14 wherein said strap of the hand part has an enlarged far end.

19. A hand orthosis according to claim 17 wherein said strap of the hand part has an enlarged far end which is larger than said ring or loop and is compressible or foldable for passing said enlarged end through said ring or loop.

20. A hand orthosis according to claim 1 wherein the hand part and forearm part are integrally formed from a heavyweight flexible fabric material.

21. A hand orthosis according to claim 1 where in the hand part and forearm part are integrally formed from a flexible planar foam material.

22. A hand orthosis according to claim 1 wherein the forearm part comprises an adjustment device for adjusting the operative length of the flexible connecting element between the forearm part and the thumb part.

23. A hand orthosis according to claim 22 wherein the adjustment device includes a bolt slidably moveable longitudinally of a barrel on the forearm part and to which bolt the flexible connecting element is attached, with a laterally extending lever enabling movement of the bolt for adjustment of the length of the operative portion of the flexible connecting element and locking of the bolt in a multiple number of selected positions in said barrel.

24. A hand orthosis comprising;
a thumb part attachable to a user's thumb
a hand part for fittable around a user's hand between the thumb and knuckles,
a forearm part for fittable to a user's forearm above the hand, and
a connecting element connected to the thumb part and to another part of the orthosis,
a power unit to which the connecting element is also connected, and
a control device operable by the user to cause the power unit to via the connecting element draw the user's thumb towards the user's index finger to form a grip

25. A hand orthosis according to claim 24 wherein the hand part is fitable to a user's hand without insertion of the user's fingers into the orthosis.

26. A hand orthosis according to claim 24 wherein the forearm part comprises one or more fasteners for releasably securing the forearm part around a user's forearm.

27. A hand orthosis according to claim 26 wherein the one or more fastener(s) enable the forearm part to be opened so that the forearm part can be fitted to or removed from a user's forearm without requiring sliding of the user's forearm part through the forearm part.

28. A hand orthosis according to claim 27 wherein the one or more fastener(s) of the forearm part is/are on the outside of the user's forearm when the orthosis is worn.

29. A hand orthosis according to claim 28 wherein the forearm part comprises a major section which extends longitudinally of the user's inside forearm when the orthosis is worn, and at least one strap attached thereto which extends from one side to an opposite side of said major section around the back of the user's forearm.

30. A hand orthosis according to claim 29 wherein when the orthosis is worn said strap(s) can pass from one side of said major section and around the back of the user's forearm to said opposite side of the major section of the forearm part, and then double back on itself/themselves.

31. A hand orthosis according to claim 30 comprising one or more rings or loops attached to said opposite side so that before doubling back the strap(s) may pass through a ring or loop at said opposite side of the major section of the forearm part.

32. A hand orthosis according to claim 31 wherein each strap comprises sections of a hook and loop fastening material by which a far end of the strap may be attached back to itself.

33. A hand orthosis according to claim 24 wherein said thumb part is in the form of C-shaped clip formed of a resiliently flexible material.

34. A hand orthosis according to claim 24 wherein the hand part comprises a palm section which when the orthosis is worn extends at least partly over the palm of the user's hand, and a strap which can extend from one side of said palm section around the back of the hand to another side of the palm section to encircle the hand.

35. A hand orthosis according to claim 34 wherein said strap of the hand part may pass from one side of said palm section, around the back of the user's hand, and to said another side of said palm section and then double back on itself.

36. A hand orthosis according to claim 35 comprising a ring or loop attached to said another side of said palm section so that before doubling back on itself the strap may pass through said ring or loop.

37. A hand orthosis according to claim 36 wherein said strap of the hand part comprises sections of a hook and loop fastening material by which a far end of the strap may be attached back to itself.

38. A hand orthosis according to claim 24 wherein the hand part and forearm part are integrally formed from a heavyweight flexible fabric material.

39. A hand orthosis according to claim 24 wherein the hand part and forearm part are integrally formed from a flexible planar foam material.

40. A hand orthosis according to claim 38 also including a splint incorporated in the orthosis to strengthen the orthosis against bending at the wrist when worn.

41. A hand orthosis according to claim 24 wherein in lower unit includes a pneumatic ram.

42. A hand orthosis according to claim 41 wherein the lower unit also includes an air cylinder for storage of air under pressure.

43. A hand orthosis according to claim 24 wherein said connecting element is a Bowden cable.

44. A hand orthosis comprising;
a thumb part attachable to a user's thumb
a hand part fittable to a user's hand without insertion of the user's fingers into the orthosis,
a forearm part fittable to a user's forearm above the hand, and
a flexible connecting element connected at one end to the thumb part and to another part of the orthosis and operable by a user to draw the user's thumb towards the user's index finger for form a grip.
